(19) United States
(12) Patent Application Publication
(10) Pub. No.: US 2005/0069509 A1
(43) Pub. Date: Mar. 31, 2005

(54) Oxygen rich scalp treatment

(76) Inventor: Francis Busch JR., Southbury, CT (US)

Correspondence Address: THEODORE C. JAY, APARTMENT 600, 16 NORTH CHATSWORTH AVE., LARCHMONT, NY, 10538, US

(21) Appl. No.: 10/675,871

(22) Filed: Sep. 30, 2003

(51) Int. Cl. A61K 7/06; A61K 33/00

(52) U.S. Cl. 424/70.1

(57) ABSTRACT

Method for Application of oxygen to the scalp from cosmetic shampoo with pH adjusted between 8.0 and 11.0 and hair conditioning cream containing from 1.0% to 4.0% hydrogen peroxide. The shampoo first used is not completely rinsed leaving the pH of the scalp between 8.0 and 11.0. The hydrogen peroxide containing cream rinse is the applied to the scalp and hair and left on the hair for at least on minute. Pure oxygen is released directly into the scalp supporting the health of the hair and scalp.

### FIELD OF THE INVENTION

This invention relates to the application of dissolved oxygen to the scalp released from a shampoo and hair conditioner used in combination.

### BACKGROUND OF THE INVENTION

Oxygen is an essential element and supports the life of all cells including those of the scalp and hair follicle. While a majority of human oxygen consumption comes from lung function where it is absorbed into the blood stream, a significant amount comes directly through the skin. Skin Respiration is the biological description of this breath like function.

It is well understood that that the health of the hair and scalp depend on adequate oxygen availability, the oxygen being supplied both by lung function and skin respiration. The present invention is directed toward enhancing the amount of oxygen supplied as skin respiration applied directly to the scalp and hair as where it becomes available supporting the health and appearance of both.

### BRIEF SUMMARY OF THE INVENTION

It is an object of the present invention to increase the health and appearance of the hair and scalp by supplying oxygen directly to the wet scalp and hair.

It is a further object to supply dissolved oxygen to the wet scalp and hair a shampoo having a raised pH and a conditioner containing hydrogen peroxide.

Yet another object is to increase the diameter of hair by a substantial percentage by supplying, to the wet scalp and hair, a shampoo having a raised pH and a conditioner containing hydrogen peroxide.

These and other objects of the invention will either be explained or will become apparent hereinafter.

The present invention is directed toward a method for supplying oxygen directly to the scalp by first wetting the scalp. Secondly, a shampoo formulation is applied to the scalp. This formulation provides gentle cleansing of the scalp and hair as is typical of a well formulated shampoo. However, this formulation also contains a suitable base which raises the pH of the scalp and hair. Finally, a conditioner containing hydrogen peroxide is applied to the scalp and hair. It is well known that oxygen is released from hydrogen peroxide according to the following chemical equation: 2H₂O₂→2 H₂O+agent or a bleaching agent. The increased pH facilitates the quick release of oxygen from hydrogen peroxide contained in the conditioner of this invention. Since the process of washing and conditioning hair occurs in a very brief period of time, reaction time is of critical importance.

The oxygen supplied to the scalp is dissolved oxygen and is carried by water. When the skin is wet, it immediately absorbs up to 500 times its weight in water. It is this water that carries the oxygen into the scalp supporting the skin respiration process.

The benefits of oxygen released in this inventive process, surprisingly have increased the diameter of hair so treated. After a first treatment with the test regimen, average hair diameter increased by 9.2%. With multiple treatments, the average hair diameter increased further, up to 16.4%. Moreover, hair so treated was evaluated by a curl retention method, wherein after one cycle, curl retention was increased by 90.4% and after five cycles 155.2% as compared to a conventional water treatment used as a control.

### DETAILED DESCRIPTION OF THE PRESENT INVENTION

The shampoos of this invention contain surfactant ingredients that are stable at pH levels ranging between 8.0 and 11.0. These ingredients are well known to those skilled in this art and brief descriptions of each type may be found in HARRY'S COSMETICOLOGY 8^{TH} Edition, Chemical Publishing Co., Inc., New York, N.Y.

The preferred surfactants used in the shampoos of this invention are blends of non-ionic, amphoteric and anionic compositions. The pH of the shampoo formulations of this invention is adjusted to between 8.0 and 11.0 with base materials that include sodium hydroxide, potassium hydroxide and sodium bicarbonate. Sodium bicarbonate is the preferred base used to adjust the pH of the shampoos of this invention. The shampoos of this invention further contain preservatives, water, hair softening materials and fragrances typical of those well known in the art. The viscosity of the shampoos of this invention can be increased using non-ionic thickening agents well known to those skilled in the art.

### SPECIFIC EXAMPLES OF THE SHAMPOOS OF THIS INVENTION

| Shampoo: | Example A | Example B |
|---|---|---|
| Ingredient deionized water [parts by weight | 47.20 | 45.2 |

PRESERVATIVES

| | | |
|---|---|---|
| Kathon | .05 | 05 |
| Methylparaben | .10 | .10 |
| Propylparaben | .10 | 10 |
| Na₂ EDTA | .05 | .05 |

SURFACTANTS

| | | |
|---|---|---|
| Non-Ionic | | |
| Cocamidopropylamine oxide | .60 | .60 |
| Amphoteric | | |
| Cocoamidopropyl Betaine | 12.0 | 12.0 |
| Anionic | | |
| Sodium C14-16 Olefin Sulfonate | 25.0 | 25.0 |

NON-IONIC THICKENER

| | | |
|---|---|---|
| Ceteareth-60 Myristyl Glycol | 13.0 | 13.0 |
| Fragrance | .2 | .2 |

PH ADJUSTER

| | | |
|---|---|---|
| Sodium Bicarbonate | 0.50 | 2.0 |

CONDITIONING AGENTS

| | | |
|---|---|---|
| DL Panthanenol | q.s. | q.s. |

### SPECIFIC EXAMPLES OF THE CONDITIONING AGENTS OF THIS INVENTION

The conditioning agents of this invention are formulated to provide a source of hydrogen peroxide to the scalp which becomes dissolved oxygen according to the above stated chemical equation. It is well known by those skilled in the art to choose hair conditioning ingredients which remain stable in the presence of hydrogen peroxide. The following preferred examples are oil in water emulsions and contain non ionic emulsifying agents and conditioning hair conditioning agents.

| PEROXIDE CONDITIONING AGENT | Example A | Example B |
|---|---|---|
| Water (Aqua) | 85.3 | 85.3 |
| Citric Acid | 2.0 | 2.0 |
| Methylparaben | 0.2 | 0.2 |
| Imidazolene Urea | 0.5 | 0.5 |
| Cetearyl Alcohol (and) | 1.5 | 1.5 |
| Ceteareth-20 | | |
| Cetearyl Alcohol | 1.5 | 1.5 |
| Glycerol Monostearate | 0.75 | 0.75 |
| Glycerol Di-Stearate | 0.75 | 0.75 |
| Triceteareth-4 Phosphate | 0.5 | 0.5 |
| Propylparaben | 0.25 | 0.25 |
| Dimethicone | 0.5 | 0.5 |
| Myristyl Octanoate | 4.5 | 4.5 |
| Cetyl Alcohol | 1.0 | 1.0 |
| D L Panthenol | 0.5 | 0.5 |
| Fragrance (Parfum) | q.s. | q.s. |
| Dowicil | 0.25 | 0.25 |
| Hydrogen Peroxide | 1.0 | 2.0 |

### TEST RESULTS

The pH of the above Example A and B shampoos was measured using an ORION pH METER Model 210 calibrated with certified buffers.

Results pH test:

Example A shampoo 8.0
Example B shampoo 11.0
   The dissolved oxygen was measured using a HANNAH Dissolved Oxygen meter Model 210. Samples were prepared for oxygen measurement according to the following formula:
   1 part of Example A Shampoo, 15 Parts Example A conditioner and 16 parts distilled water. Test Solution A
   1 part of Example B Shampoo, 15 parts Example B conditioner and 16 parts distilled water. Test Solution B

The test solutions thus prepared created a pH for the test solution similar to first wetting the hair and scalp washing the hair with shampoo, followed by rinsing shampoo and application of the peroxide hair conditioner of this invention. A control was prepared for each test conditioner by replacing the one part test shampoo of the above test solutions with one part of distilled water and then mixing with an additional 16 parts of distilled water.

| | pH of test solutions |
|---|---|
| Test solution A | 8.0 |
| Test solution B | 11.0 |
| Test solution Control A | 3.5 |
| Test solution Control B | 3.5 |

The pH of this control measured 3.5 and the dissolved oxygen 4.90 ppm after one minute and 1.56 ppm after 10 minutes.

### OXYGEN CONTENT PPM

| | 0 MIN | 1 MIN | 2 MIN | 3 MIN | 4 MIN | 5 MIN | 10 MIN | 15 MIN |
|---|---|---|---|---|---|---|---|---|
| CONTROLS A&B | 4.90 | 3.10 | 2.29 | 2.01 | 1.92 | 1.78 | 1.56 | |
| EXAMPLE A | 11.00 | 10.38 | | | | 8.92 | 8.28 | 8.04 |
| EXAMPLE B | 15.2 | 15.0 | | | | 10.5 | 10.0 | 9.0 |

The above experiment was repeated with the shampoo and conditioner of Example B. The resulting pH measured 8.7 and ppm of dissolved oxygen 11.0 and 15.0 ppm after one minute and 10.0 ppm dissolved oxygen after 10 minutes.

From the above test it is clear that oxygen is released from the conditioners of this invention when the pH is first elevated.

While the invention has been described with particular reference to the specific examples, the protection sought is to be limited only by the terms of the claims that follow.

1. A method for increasing the health and appearance of the hair and scalp by supplying oxygen directly to the scalp and hair, said method comprising:
[a] first wetting the hair and scalp;
[b] secondly, applying to the scalp and hair a cosmetic shampoo with pH adjusted between 8.0 and 11.0; and
[c] thirdly applying to the scalp and hair a conditioning cream containing from 1.0% to 4.0% hydrogen peroxide wherein the pH of the scalp remains between 8.0 and 11.0.

2. The method of claim 1 wherein the dissolved oxygen is generated in one minute or less.

3. The method of claim 1 wherein the shampoo contains a mixture of non-ionic, amphoteric and anionic surfactants.

4. The method of claim 1 wherein the pH is adjusted with sodium bi-carbonate.

5. The method of claim 1 wherein the conditioner contains hydrogen peroxide in an amount from 1 to 3 parts by weight
